(19) United States
(12) Patent Application Publication
(10) Pub. No.: US 2014/0025179 A1
(43) Pub. Date: Jan. 23, 2014

(54) BRITTLE BIOCOMPATIBLE COMPOSITES AND METHODS

(71) Applicants: Arthur J. Fortini, Pasadena, CA (US); Richard Kaplan, Pacoima, CA (US); Gregory Borwick, Reseda, CA (US)

(72) Inventors: Arthur J. Fortini, Pasadena, CA (US); Richard Kaplan, Pacoima, CA (US); Gregory Borwick, Reseda, CA (US)

(73) Assignee: Ultramet, Pacoima, CA (US)

(21) Appl. No.: 13/829,858

(22) Filed: Mar. 14, 2013

Related U.S. Application Data
(60) Provisional application No. 61/674,117, filed on Jul. 20, 2012.

(51) Int. Cl. A61F 2/28; B32B 3/26

(52) U.S. Cl. CPC A61F 2/28; B32B 3/26
USPC 623/23.5; 428/304.4; 428/312.8; 623/23.51; 427/2.26

(57) ABSTRACT

Reticulated composites are formed with a thin coating of a ductile biocompatible metal such as tantalum on a brittle biocompatible substrate such as vitreous carbon. Such composites exhibit physical properties that permit these monolithic composites to be morselized, or sized and shaped manually. Such composites exhibit surfaces with excellent ductile metal biocompatibility properties, but with strength and fracture physical properties that are more characteristic of the brittle substrate than of the ductile metal. Such composites fracture easily, and may be morselized or worked by manual sizing and shaping. Such morselization and working is accomplished by breaking or fracturing the composite, rather than plastically deforming it. Morselized or manually shaped reticulated composites exhibit excellent biocompatibility characteristics with micro- and nano-textured surfaces that promote rapid bone ingrowth and adhesion. When the composite is broken, the substrate is exposed. The presence of exposed substrate does not significantly impair the biocompatibility of these composites.

### RELATED APPLICATIONS

This application claims the benefit U.S. Provisional Application No. 61/674,117, filed Jul. 20, 2012.

### TECHNICAL FIELD

This invention relates to the field of particulate and monolithic orthopedic biocompatible composites for use as implant replacements for bone, and their manufacture and use.

### BACKGROUND

Various synthetic biocompatible composite materials had been previously proposed for use as orthopedic biomaterials. It had been proposed that these materials should serve as structures into which new bone may grow.

Reticulated composites formed by the chemical vapor deposition (CVD) of tantalum metal on reticulated carbon foam have long been used for in vivo implantation. See Kaplan U.S. Pat. No. 5,282,861, which is hereby incorporated herein by reference as though fully set forth hereat. Such reticulated composite materials have been sold for orthopedic uses under the designation, “Trabecular Metal.” Trabecular Metal is a trademark of Zimmer, Inc. The carbon substrates generally comprised an open network of ligaments joined together at nodes. See, for example, FIG. 1. The tantalum generally conformed to and fully encapsulated the carbon substrate in previous reticulated composites. Tantalum typically occupied between approximately 15 and 20 volume percent of the bulk volume of the implant, with the balance being approximately 3 volume percent carbon substrate, and approximately 77 to 82 volume percent interconnected void space. See, for example FIGS. 2, 3, and 14. In such reticulated composites the strength and ductility of the composites were approximately the same as those of the tantalum coating. The carbon substrate contributed very little to the strength and ductility properties in those composites. The carbon substrates were generally vitreous carbon. Conventionally, reticulated carbon foam had been coated with ductile tantalum in such an amount that the strength and ductility properties of the ductile tantalum dominated the properties of the resulting composite. See, for example, FIG. 2 and FIG. 3. The resulting reticulated tantalum foam implant was strong and deformed rather than fracturing when subjected to a load.

Conventionally, tantalum deposits were generally formed under conditions where elements that might cause embrittlement of the tantalum were kept to a low concentration in the deposit to maintain the highest degree of ductility. A small amount of other elements may have been included in such coatings for the purposes of strengthening or otherwise improving the properties of the pure tantalum. The resulting tantalum deposits were both ductile and strong, as well as being very fracture resistant and biocompatible. These properties of ductility, fracture resistance, and strength had previously been believed to be required of a reticulated tantalum-carbon composite that was intended for an orthopedic use. These properties allowed the reticulated composite to be used as a structural replacement for natural bone where a load bearing structure was desired.

The tantalum coating process conventionally involved, for example, chemical vapor infiltration (CVI) procedures carried out to form a completely enveloping deposit of tantalum on reticulated carbon skeletons. CVI is a form of chemical vapor deposition (CVD) wherein the deposited metal is infiltrated into a porous body, and deposited on the interior surfaces of that body. CVI procedures were used to deposit the tantalum because they resulted in the production of a closely controlled tantalum deposit that reliably exhibited highly desirable physical and biocompatible surface properties. See, for example, the enlarged portion of FIG. 3 for a visual description of a nano-textured, ductile, tantalum surface that exhibited exceptionally good biocompatibility. The term “nano” as used herein means less than 1 micron for purposes of this disclosure and the attached claims. These surface properties encouraged rapid bone ingrowth into a reticulated implant. These surface properties also promoted good adhesion between the new in-grown bone and the reticulated implant. The risk of the patient's body rejecting the resulting implant was minimized. Reticulated tantalum composite structures were osseointegrated rapidly and effectively in the human body.

The incorporation of small amounts of impurities, such as, for example, oxygen, into the tantalum deposit on the brittle reticulated substrate results in a slightly less ductile but generally stronger tantalum deposit. Amounts of impurities in excess of these small amounts may degrade the physical and surface properties of the tantalum to such a degree that it was previously believed to be unsuitable for implantation purposes. Brittle tantalum deposits could result from the presence of impurities during formation of the tantalum deposit or during subsequent elevated temperature processing in the presence of embrittling agents such as hydrogen. Such degradation of physical properties was generally regarded as undesirable, because it impaired the strength and load bearing properties of the resulting implant.

Tantalum deposits that were conventionally laid down by chemical vapor infiltration (CVI) processes during the formation of ductile tantalum composites exhibited micro- and nano-textured surfaces. Under moderate magnification, the prior micro-textured surfaces generally exhibited features including a chaotic jumble of angular terraces, blocks, facets, and walls projecting from the surface of the tantalum coating. The individual elements of the features, on average, usually had peak to valley heights of approximately 1 to 5 microns. See, for example, FIG. 6 or FIG. 3. Under higher magnification, the surfaces of the individual elements exhibited approximately regular nano-patterns of terraces, walls, and newly nucleated crystals. See, for example, FIG. 7. These nano-patterns generally projected from the surface of the individual elements by approximately 300 to 500 nanometers. See, for example, the enlarged area in FIG. 3. There are certain applications where the characteristic strength and ductility of conventional reticulated tantalum precludes, or at least substantially restricts its use. For example, sometimes the particular shape and size requirements of an implant are not known until surgical procedures have exposed the site where the implant is to be inserted. The surgeon must be able to shape and size the implant during the surgery to fit the needs of the patient. Because of the necessity to maintain certain operating room conditions, and finish the surgical procedure within certain time constraints, the surgeon must be able to shape and size an implant manually with just the tools that are normally available in an operating room.

The characteristic strength and ductility of conventional tantalum foam are such that it is very difficult, if not impossible, for a surgeon to manually shape and size it during an operation. Conventional reticulated tantalum undergoes ductile deformation, that is, it smears instead of fracturing. Generally, more force than can usually be applied manually is required to cut a conventional reticulated tantalum implant. The machines and procedures that are normally used to shape reticulated tantalum objects without deforming or smearing them are not suitable for use during surgery in an operating room environment.

It had been previously proposed to manufacture small tantalum flakes from a thin ductile sheet of tantalum for use in electrical capacitors. According to a previously proposed manufacturing procedure, thin tantalum sheets were hydrided to form a brittle tantalum or tantalum hydride foil, the brittle foil was reduced to small flakes by milling, and the resulting brittle flakes were rendered ductile by dehydriding them. See McCracken et al. US 2008/0233420, published Sep. 25, 2008, which is hereby incorporated herein by reference as though fully set forth hereat.

It had also been previously proposed to use open-cell metal structures as implants, and to shape them immediately before or during an operation. See Nies US 2010/0185299, published Jul. 22, 2010, which is hereby incorporated herein by reference as though fully set forth hereat. No method of shaping the metal structures is proposed, suggested, or taught by Nies.

It had further been previously proposed that separate structural elements in the shape of caltrops could be interlocked with one another in an array so the array could resist shear stress in essentially all directions. See Black et al., U.S. Pat. No. 5,676,700, patented Oct. 14, 1997, which is hereby incorporated herein by reference as though fully set forth hereat. No method for forming the caltrop shape is proposed, suggested, or taught by Black.

Reticulated brittle substrates, of the general type shown in FIG. 1 had been previously proposed for use in ductile reticulated tantalum composite implants. See also, for example, Stankiewicz U.S. Pat. No. 6,103,149, which is hereby incorporated herein as though fully set forth hereat. This patent described embodiments of reticulated vitreous carbon foam that were proposed for use as a substrate in reticulated tantalum composite implants. Reticulated carbon foam made according to this patent, or otherwise, is very brittle. It deforms very little before fracturing when loaded in any of tension, compression, or shear, and it exhibits very little fracture toughness. Vitreous carbon, the material of which carbon foam is conventionally comprised, exhibits good biocompatibility properties. Stankiewicz teaches the production of reticulated carbon skeletons containing generally spheroidal pores with aspect ratios between approximately 0.8 and 1.2 from polymeric foam. The spheroidal pores may be made more prolate or oblate, and the porosity of the skeletons decreased by compressing the polymeric foam.

In the event of any inconsistencies, contradictions, or contra indications between the teachings herein and the teachings of any reference that is incorporated herein by reference, the teachings herein shall control.

### BRIEF SUMMARY OF THE INVENTION

Tantalum has been used as an orthopedic biomaterial in reticulated implants at least in part because of its strength and ductility. It is a very good load bearing metal. Also, it is very biocompatible. When properly prepared, CVD tantalum exhibits a micro- and nano-textured surface that promotes bone ingrowth and adhesion. Other ductile metals, such as, for example, niobium and titanium have also been used as orthopedic biomaterials. Because of its strength and ductility, tantalum has not been used where morselization or manual shaping of a monolith is required.

According to embodiments of the invention, reticulated monolithic tantalum and other biocompatible metal composites exhibit physical properties that permit these monolith composites to be morselized, or sized and shaped manually. Certain reticulated tantalum and other biocompatible metal composite embodiments exhibit surfaces with characteristic ductile metal biocompatibility properties, but with strength and fracture physical properties that are more characteristic of the brittle substrate than of the ductile metal. Such embodiments fracture easily, and may be morselized or worked by manual sizing and shaping. In further embodiments certain morselized composites exhibit caltrop shaped configurations. Morselized or manually shaped monolithic composites exhibit excellent biocompatibility characteristics with micro- and nano-textured surfaces that promote rapid bone ingrowth and adhesion. Additional embodiments may involve applying additional tantalum or other materials to the morselized composites to alter their physical or biocompatibility properties. Such additional deposits may, for example, fully encapsulate the brittle substrate, alter the proportion of ductile tantalum in the composite sufficiently to influence the strength and ductility of the morselized composite, or add other materials that improve the biocompatibility of the composites or promote bone ingrowth.

Monolith composites may be shaped by a surgeon during surgery. Embodiments are brittle enough and/or have a low enough strength that a surgeon can shape them by hand during surgery using only the tools that are normally available in an operating room environment. These include hand carving, chiseling, and sawing tools as well as small hand-held grinders, saws, drills, Dremel tools (“Dremel” is a registered trademark of Robert Bosch Tool Corp.), and other power tools. Certain embodiments are brittle enough that they may be manually reduced in size and shaped by fracturing. Such embodiments primarily fracture rather than undergoing plastic deformation. The fractured material may generally be removed, and the remaining custom-shaped monolith may be implanted in a patient.

Reticulated tantalum and other biocompatible composites intended for use as surgeon shapeable implants include a brittle, biocompatible, reticulated substrate, with a small controlled amount of ductile tantalum or other ductile metal deposited on the surface of the substrate. There is enough tantalum or other metal in the surface deposit on the brittle substrate to provide a biocompatible micro- and nano-textured surface that promotes the ingrowth and adhesion of new bone. The tantalum or other metal deposit does not include enough tantalum or other metal to prevent the reticulated composite from fracturing. When worked, the reticulated tantalum or other metal composite primarily undergoes fracturing rather than plastic deformation, thus allowing it to be manually shaped by a surgeon with the tools that are normally available in an operating room. In certain embodiments the reticulated composite undergoes some degree of plastic deformation in addition to fracturing, but not enough to render it manually unworkable.

The amount of tantalum or other metal contained in conventional reticulated composites is such that the metal generally determines the machinability, and at least the mechanical properties of strength, fracture toughness, and ductility. The brittle properties of the substrate are able to largely dominate the physical fracture, strength and ductility properties of the reticulated tantalum or other metal composite only when the metal content is substantially lower than in conventional reticulated tantalum composites. Even when the tantalum or other volume percent is so low that the substrate dominates the physical properties of the reticulated composite, the biocompatible, ingrowth, and adhesion properties of such a composite will be dominated by the surface properties of the thin tantalum or other metal coating. Thus, a surgeon will be able to utilize a substantially monolithic implant that is physically shapeable in the environment in which the surgeon must work, and yet will exhibit substantially all of the superior biocompatibility properties that are normally associated with ductile tantalum or other metal implants. Very thin ductile tantalum coatings contribute some strength and ductility to such reticulated tantalum composites, but not so much strength or ductility or fracture toughness that it will prevent the material from being manually shaped in the operating room.

Certain embodiments of a method of manufacturing comprise selecting a skeleton that is reticulated, biocompatible, and substantially monolithic. The skeleton comprises a reticulated three-dimensional network of ligaments interconnected at nodes to define pores and windows between said pores. The skeleton has from approximately 3, or 10, or 20, or 40 to 300, or 200, or 150, or 100 pores per inch, a fracture strength in compression of less than approximately 150 pounds per square inch, and a void volume of at least approximately 66 percent. A deposit of ductile biocompatible metal is formed on the skeleton in an operation that comprises practicing a chemical vapor infiltration procedure, for example as described by Kaplan U.S. Pat. No. 5,282,861, to form a reticulated composite. The operation is carried out under conditions wherein the deposit exhibits a surface adapted to promote bone ingrowth and adhesion. The deposit has a stand-alone deformation tensile strength of at least approximately 20,000 pounds per square inch. The deposit is limited to an amount at which the reticulated composite has a fracture strength in compression of less than approximately 1000, or 1200, or 2000 pounds per square inch. The deposit may comprise tantalum, or niobium, titanium, or other biocompatible metals, or their alloys.

Certain embodiments of a method of using a reticulated composite monolith comprise commencing an in vivo surgical procedure on a living being that will require that a portion of the living being's bone be replaced by a customized scaffold. A reticulated composite monolith that comprises a brittle biocompatible substrate and a ductile metal coating that is biocompatible and has a 3-dimensional network of ligaments interconnected at nodes forming pores and windows between said pores with pore diameters ranging from approximately 400 to 7,500 microns, or from approximately 400 to 900 microns is selected. The windows range from approximately 30 to 4500 microns in diameter, or from approximately 30 to 400 microns in diameter. The reticulated composite monolith has substantially no deformation strength, a fracture strength in compression of less than approximately 1000, or 1200, or 2000 pounds per square inch, and the biocompatible surface characteristics of a ductile metal such as, for example, tantalum. The customized scaffold is formed by manually shaping the reticulated composite monolith during the in vivo surgical procedure. The manually shaping includes fracturing the reticulated composite monolith to change its shape. Fracturing the reticulated composite monolith expose the biocompatible substrate where the reticulated composite monolith is fractured. No significant plastic deformation of the monolith takes place during this shaping operation. Fracturing of the ligaments and nodes accounts for substantially all of the changes in shape that this manual shaping step accomplishes. The substrate is exposed at the location where the ligaments or nodes are broken. The composite debris that is broken away from the monolith is generally removed before the customized scaffold is implanted.

Certain embodiments of an article of manufacture comprise a reticulated composite monolith comprised of a 3-dimensional network of ligaments interconnected at nodes to define pores and windows between the pores. The reticulated composite monolith is comprised of biocompatible materials including a substrate and a ductile metallic coating on the substrate. The reticulated composite monolith has a fracture strength in compression of less than approximately 1000, or 1200, or 2000 pounds per square inch, and said ductile metal coating having surface characteristics that promotes bone growth and adhesion.

Certain embodiments of an article of manufacture include a reticulated composite monolith that comprises a skeleton of vitreous carbon coated with from approximately 1 to 10 volume percent of ductile tantalum, or from approximately 2 to 5 volume percent ductile tantalum.

Certain embodiments of a reticulated composite monolith that is biocompatible comprise a skeleton. The skeleton comprises vitreous carbon, and is substantially monolithic. The skeleton comprises a reticulated three-dimensional network of ligaments interconnected at nodes to define generally spheroidal pores and windows between the spheroidal pores. The spheroidal pores range in diameter from approximately 400 to 7500 microns, or between approximately 400 and 900 microns. The windows range from approximately 30 to 4500 microns in diameter, or between approximately 30 and 400 microns. The skeleton has a stand-alone fracture strength in compression of less than approximately 150 pounds per square inch. A metal coating on the skeleton comprises ductile biocompatible metal. The metal coating has a stand-alone deformation tensile strength of at least approximately 20,000 pounds per square inch, and a submicron-textured surface that promotes ingrowth and adhesion of new bone. The metal coating is present in an amount sufficient to provide the textured surface, and proportioned relative to the skeleton such that the reticulated composite monolith has a fracture strength in compression of less than approximately 1000, or 1200, or 2000 pounds per square inch.

According to further embodiments, a composite caltrop that is biocompatible comprises a brittle substrate comprised of vitreous carbon having a stand-alone fracture strength in compression of less than approximately 150 pounds per square inch. A metal coating on the brittle substrate is comprised of ductile biocompatible metal. The metal coating has a stand-alone deformation tensile strength of at least approximately 20,000 pounds per square inch, and a submicron-textured surface that promotes ingrowth and adhesion of new bone. The metal coating is present in an amount sufficient to provide such a submicron-textured surface, and proportioned relative to the brittle substrate such that the caltrop has a fracture strength in compression of less than approximately 1000, or 1200, or 2000 pounds per square inch. The caltrop has peripheral surfaces, at least some of which are broken. The brittle biocompatible substrate is exposed where the peripheral surfaces are broken.

### BRIEF DESCRIPTION OF THE DRAWINGS

Further advantages of the present invention will become apparent to those skilled in the art with the benefit of the following detailed description of the preferred embodiments and upon reference to the accompanying drawings in which:

FIG. 1 is a millimeter scale image of a prior reticulated vitreous carbon skeleton that is suitable for use as a brittle substrate according to the present invention;

FIG. 2 is a millimeter scale image of a prior CVD deposited tantalum coating on a reticulated vitreous carbon substrate such as that shown in FIG. 1;

FIG. 3 is a millimeter scale image of a prior CVD deposited tantalum coating on a reticulated vitreous carbon substrate with an enlarged nano-scale image of a small area of the surface of this tantalum coating; and

FIG. 4 is a diagrammatic flow chart of certain of the steps carried out according to an embodiment in accordance with the present invention.

FIG. 5 is a millimeter scale image of various typical 3-dimensional, spiny, multi-axial particles (caltrops) formed by crushing an uncoated reticulated vitreous carbon substrate.

FIG. 6 is a micron scale image of a prior CVI deposited ductile tantalum coating (approximately 14 volume percent tantalum) on a reticulated vitreous carbon substrate showing the micro-scale roughness;

FIG. 7 is a higher magnification image of a part of the coating shown in FIG. 6 showing the nano-scale roughness;

FIG. 8 is a micron scale image of a cross-section of a ductile tantalum coating (approximately 3 volume percent tantalum) on a vitreous carbon substrate according to the present invention showing the thickness and micron scale roughness of the ductile tantalum coating;

FIG. 9 is a micron scale image of a thin ductile tantalum coating (approximately 4 volume percent tantalum) according to the present invention showing the micron scale and sub-micron scale roughness of the tantalum surface;

FIG. 10 is a micron scale image of another embodiment of a thin ductile tantalum coating (approximately 2 volume percent tantalum) according to the present invention showing the micron scale and sub-micron scale roughness of the tantalum surface;

FIG. 11 is an image of morselized particles of various sizes that were obtained by crushing a ductile tantalum coated (approximately 1 to 5 volume percent tantalum) reticulated composite according to the present invention;

FIG. 12 is an image of a prior proposed reticulated structure wherein the pores are regular in size and shape, and are angular in outline;

FIG. 13 is an enlarged image of section 13-13 in FIG. 12;

FIG. 14 is an image of a section of a billet comprising a prior CVI deposited coating of ductile tantalum (at least approximately 14 volume percent) on a reticulated vitreous carbon substrate, illustrating the part of the billet that is described as a pore or cell 10, and the part of the billet that is described as the window 12 between the pores or cells;

FIG. 15 is a stress-strain curve obtained by compressing a billet of prior reticulated ductile tantalum coated vitreous carbon with approximately 14 volume percent tantalum;

FIG. 16 depicts two stress-strain curves obtained by compressing reticulated thin ductile tantalum coated billets according to the present invention wherein the values along the x-axis is shown in inches of movement of the compressing element in the testing apparatus, which is proportional to strain;

FIG. 17 is stress-strain curve similar to FIG. 16 of three further embodiments according to the present invention; and

FIG. 18 is an image of a partially crushed cylindrical billet measuring approximately one-half by one-quarter inches, wherein this billet comprises an embodiment of a thinly tantalum coated (approximately 3 volume percent tantalum) reticulated vitreous carbon substrate according to the present invention.

FIG. 19 is an image of typical caltrop shaped particles obtained from machining a billet of reticulated ductile tantalum coated (approximately 1 percent tantalum) vitreous carbon on an end mill.

### DETAILED DESCRIPTION OF THE PREFERRED EMBODIMENTS

As required, detailed embodiments of the present invention are disclosed herein; however, it is to be understood that the disclosed embodiments are merely exemplary of the invention that may be embodied in various and alternative forms. Therefore, specific details disclosed herein are not to be interpreted as limiting, but merely as a representative basis for teaching one skilled in the art to variously employ the present invention.

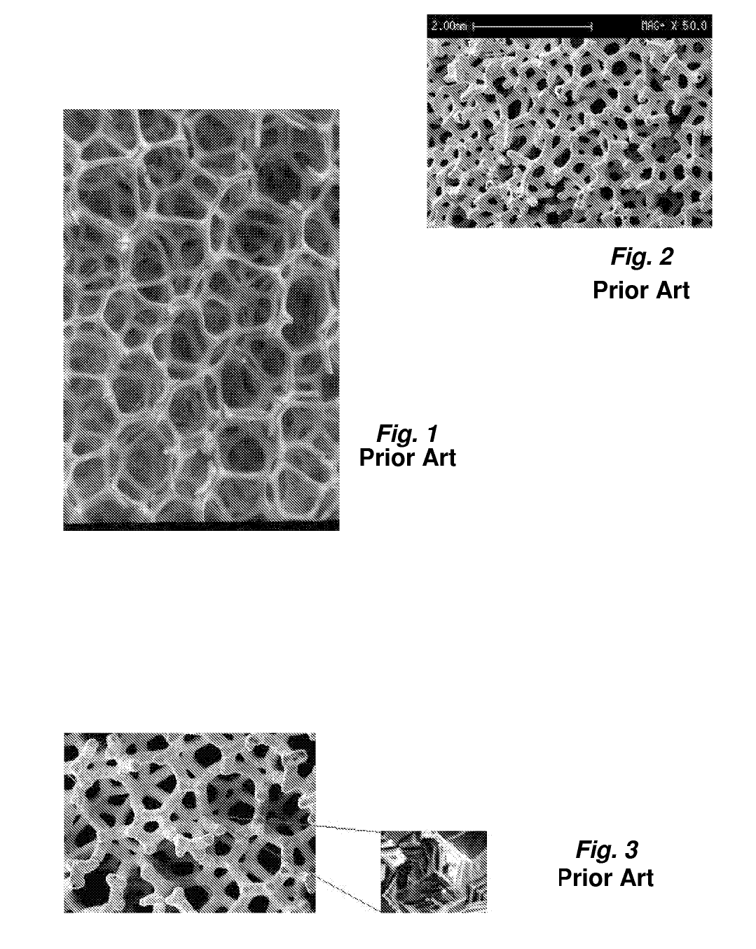

With particular reference to FIGS. 1, 2, and 3, FIG. 1 depicts a prior vitreous carbon substrate that is suitable for use in forming embodiments of the present composites. This brittle, biocompatible, reticulated skeleton exemplifies certain of the substrates that are suited for use according to the present invention. Generally, suitable substrates comprise a three-dimensional network of ligaments connected at nodes to define skeleton pores. Such suitable substrates generally exhibit low fracture toughness. That is, once a crack forms, there is little resistance to its propagation. They also have substantially no plastic deformation capability, because they break instead of deforming.

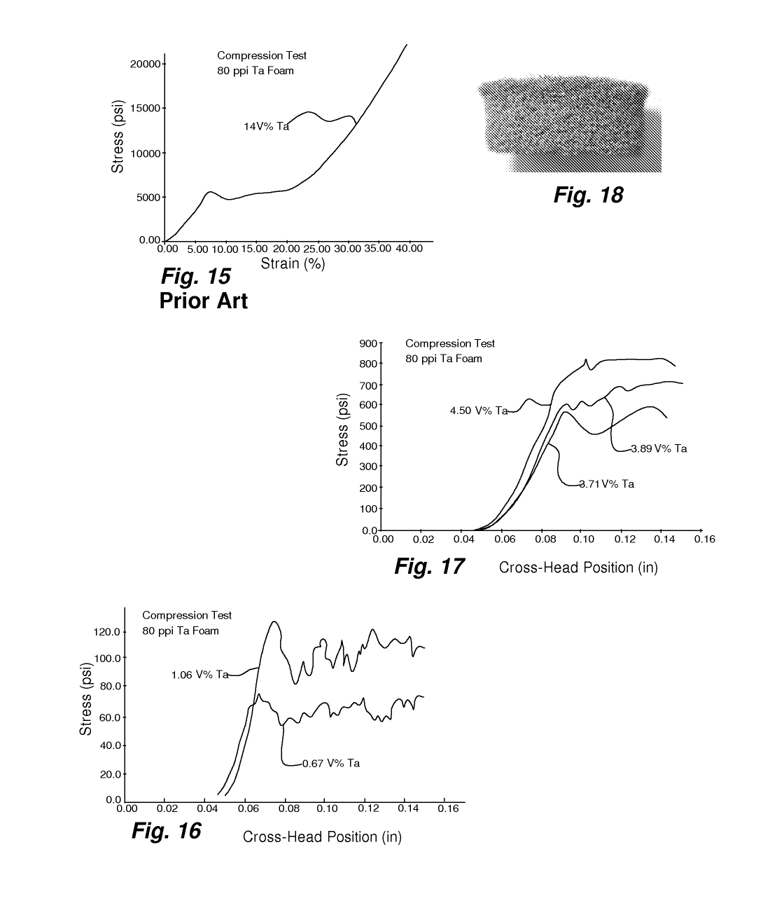

The prior reticulated tantalum-carbon composites depicted in FIGS. 2 and 3 exemplify reticulated vitreous carbon substrates that have been coated with ductile tantalum. The volume percent of ductile tantalum in such prior composites is typically at least approximately 14 volume percent. The physical properties of fracture toughness and fracture strength in compression of these composites are dominated by the physical properties of the ductile tantalum coating. These prior composites do not exhibit the brittle fracture behavior of their substrates. The stress-strain curve shown in FIG. 15 indicates the ductile behavior of these composites under compression. These conventional reticulated composite billets generally compress and flow or deform without fracturing until they become substantially pore free solid billets.

Pore sizes for generally spheroidal shaped pores in reticulated composites are often conveniently defined by their average diameters. Pores in embodiments of reticulated composites may be of any shape, not just spheroidal. Using the ordinary geometric definition of “diameter”, it is not possible to determine the diameter of a non-spheroidal shaped pore. It is, however, possible to determine the volume of a non-spheroidal pore and assign a “diameter” to it based on the diameter of a sphere with the same volume. That is, as used herein, unless otherwise indicated, “diameter,” with reference to the size of a non-spheroidal pore, is intended to mean that the volume of the non-spheroidal pore is the same as that of a sphere with the same diameter. When used herein with respect to a generally spheroidal shaped pore, “diameter” continues to have its ordinary geometric meaning. Thus, “diameter,” as used herein, unless otherwise indicated, is intended to apply to both spheroidal and non-spheroidal pores on a volume basis.

As used herein, unless otherwise indicated, “average pore size” is intended to be understood in the context of the definition herein of pore “diameter,” In reticulated embodiments, open celled porosity is generally continuous throughout the body of the composite. But for purposes of characterizing the sizes of the pores, the pores are regarded as if they were separated from one another by windows. The sizes and shapes of such windows are defined by the boundary structures that define their outlines. In reticulated embodiments, such boundary structures are typically ligaments that extend between nodes, which ligaments may be generally arcuate, generally straight, or some combination thereof. The diameters of such windows between pores are often smaller than the diameters of the pores. Such windows are generally considered to be 2-dimensional planar areas that are bounded around their edges by the structures (usually ligaments) that define their shapes. Where the pores in a reticulated composite are generally spheroidal in shape, the windows tend to be generally circular, and they have measurable average window diameters. Where such pores are non-spherodial in shape, the windows tend to be angular or irregular in shape, and they do not have diameters in the usual geometric sense of that word. Regardless of their respective shapes, all windows have some area.

It is possible to determine the area of a non-circular window in a reticulated composite, and assign a “window diameter” to it based on the diameter of a circle with the same area. That is, as used herein, unless otherwise indicated, “window diameter,” with reference to the size of a non-circular window, is intended to mean that the area of the non-circular window is the same as that of a circular window with that window diameter. When used herein with respect to a generally circular shaped window, “window diameter” continues to have its ordinary geometric meaning. Thus, “window diameter,” as used herein, unless otherwise indicated, is intended to apply to both circular and non-circular windows on an area basis. As used herein, unless otherwise indicated, “average window size” is intended to be understood in the context of the definition herein of “window diameter.”

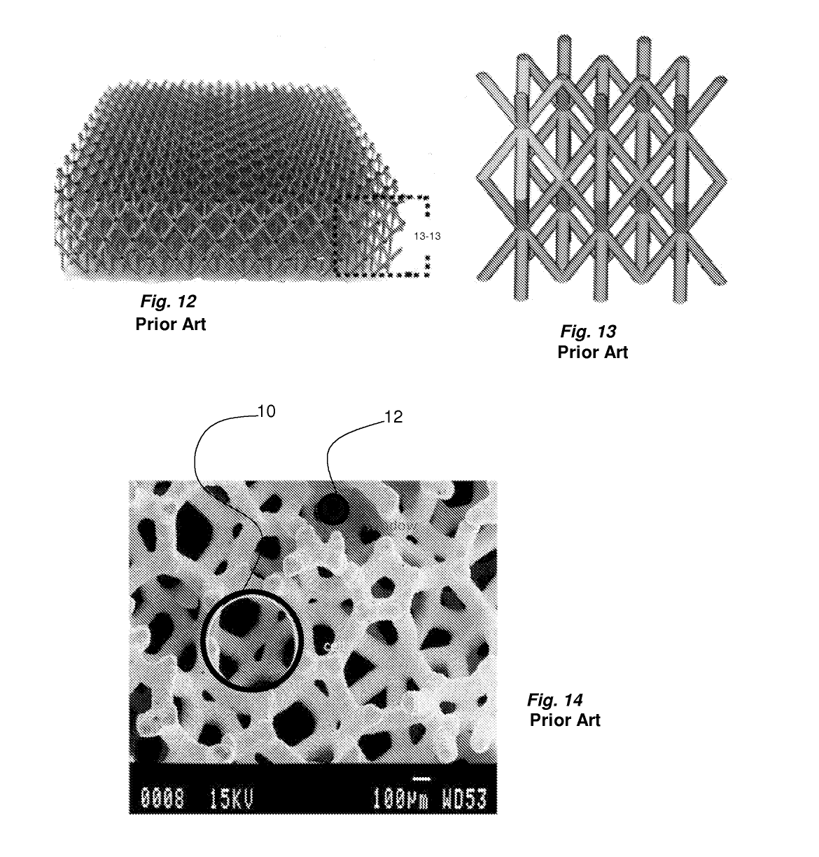

For reticulated materials comprised of straight or approximately straight ligaments, such as shown, for example, in FIGS. 12 and 13, the pores are angular, not spherodal, and the windows are not circular. For such materials, the “pore diameter” is defined in accord with the definition provided herein as the diameter of a spherical pore having the same volume as the angular pore. The window diameter is similarly defined as the diameter of a circle having the same area as the window between angular pores.

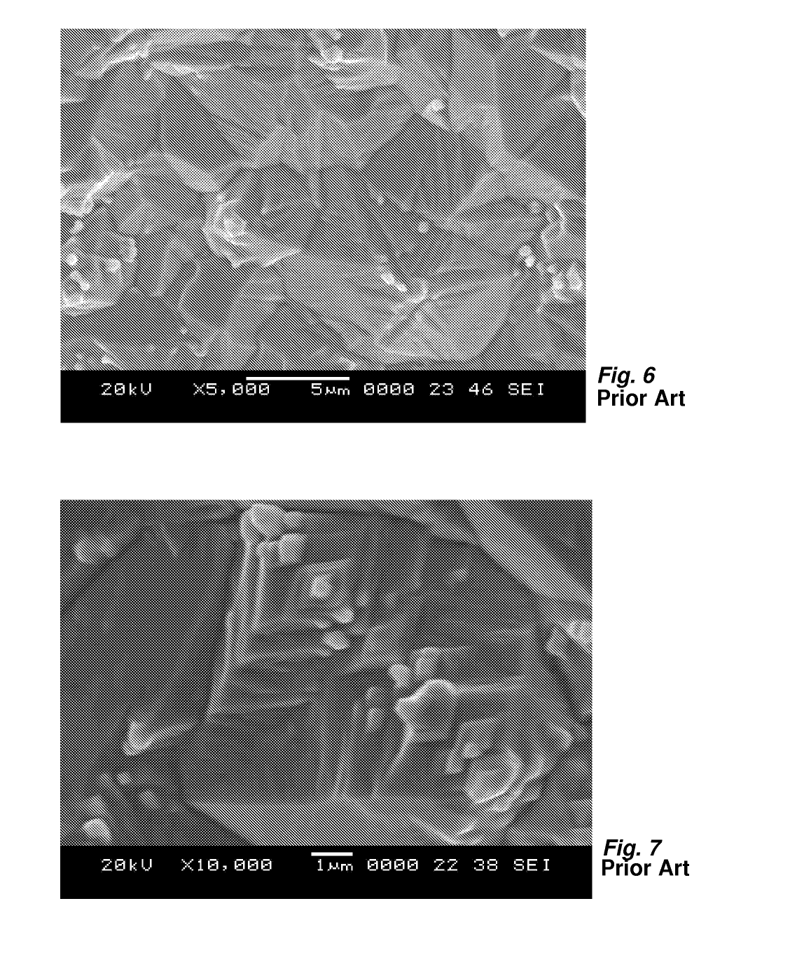

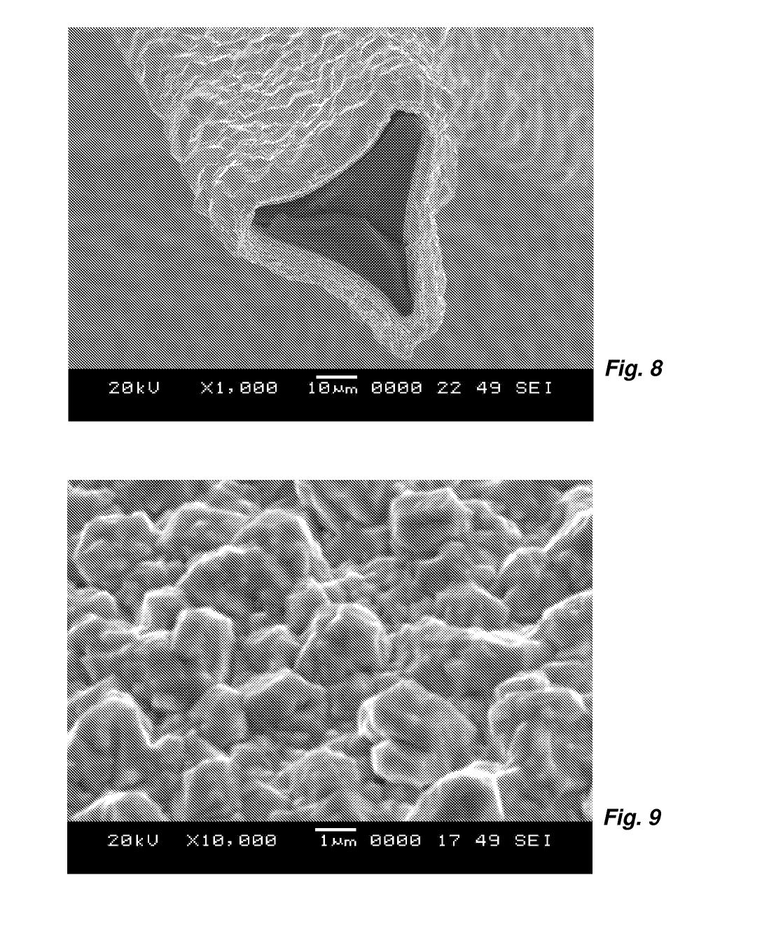

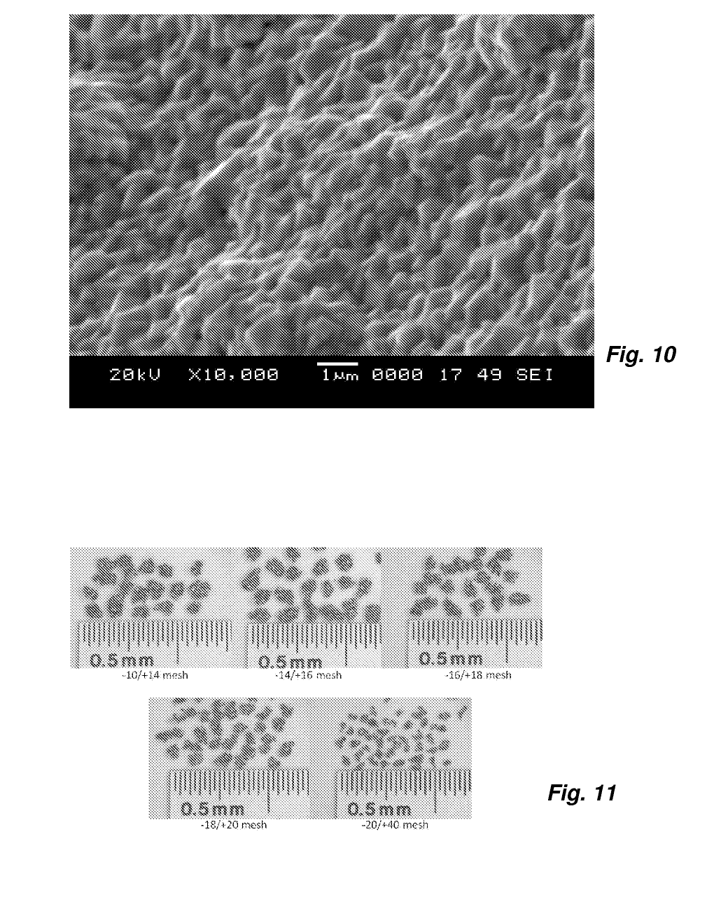

The micro texture of the surface on a conventional tantalum coating (see FIGS. 3 and 6) is such that the surface irregularities have approximately a 1 to 5 microns peak-to-valley roughness. When the tantalum coating on the carbon substrate is thinner (volume fraction, for example, of from approximately 1 to 10 volume percent tantalum, approximately 3 volume percent substrate, and the balance void volume), the micro-texture of the surface is still on the order of approximately 1 to 5 microns peak-to-valley. See, for example, FIG. 8. The nano-scale surface of such thinner tantalum coatings takes on a somewhat different morphology. See, for example, FIGS. 9 and 10 as compared to FIG. 7. The projections that form this nano-scale surface in the embodiment chosen for purposes of illustration are in the form of a chaotic jumble of sizes and shapes randomly distributed over the surface, much like boulders, cobbles, and pebbles are distributed in an unsorted glacial till, with a roughness of from approximately 1 micron to sub-0.1 microns.

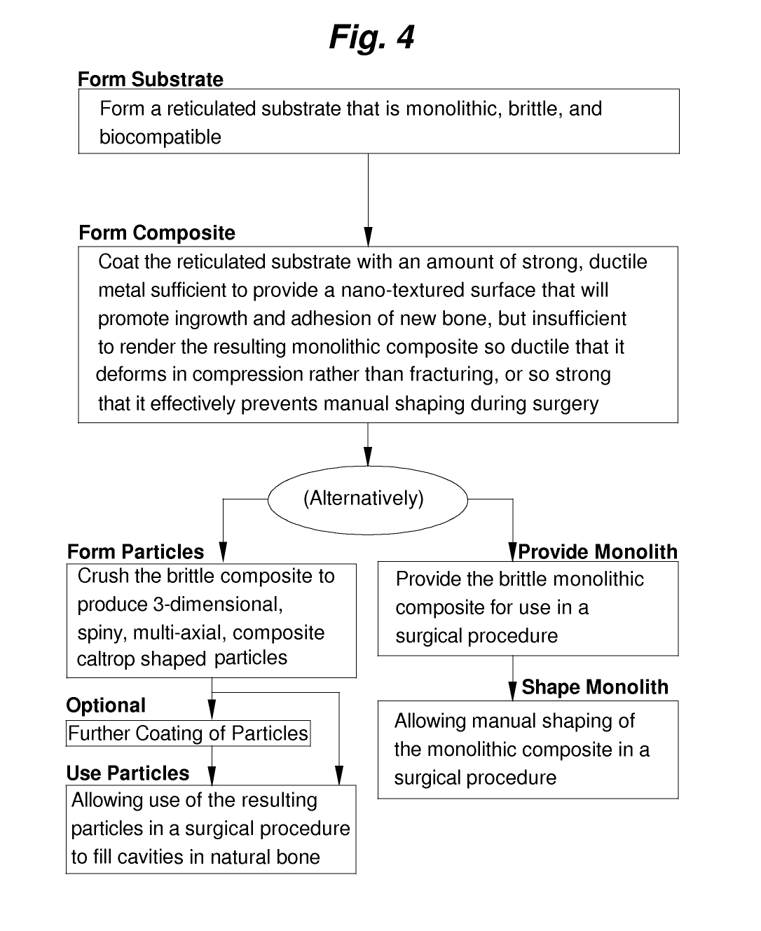

FIG. 4 diagrammatically illustrates the major steps in producing products according to the present invention. Where particles are formed the particles may optionally be further coated so as to entirely encapsulate the substrate. If this optional coating step is not employed the substrate will be exposed where the composite is fractured. No such optional coating step is possible in the alternative procedure where manual shaping takes place in the operating room. Because of this exposure the substrate should be as biocompatible as possible. Vitreous carbon, for example, is biocompatible.

The composite in FIG. 4 is characterized as being “brittle” for purposes of ease of reference. The composite must be suitable for one of its intended uses. To that end it must be workable, either by size reduction into caltrop or other useful shapes, or manual shaping of a billet. It is this workability that is intended to be captured in the term “brittle.”

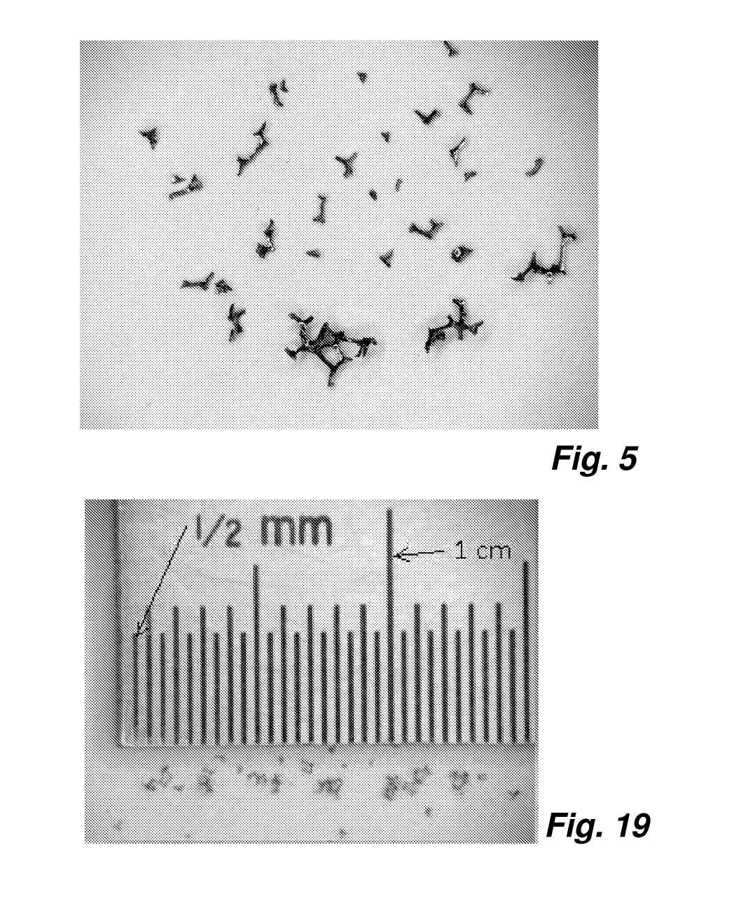

Comparison of FIG. 5 (caltrops derived from a reticulated substrate alone) and FIG. 19 (caltrops derived from a ductile tantalum coated reticulated substrate) shows that it is possible to obtain the caltrop shape in a particle with the advantageous biocompatible characteristics of ductile tantalum. If the particles shown in FIG. 19 are subjected to a further tantalum coating operation, as shown in FIG. 4, they will retain their caltrop shape even if the tantalum coating is of such an amount (for example, 14 volume percent as indicated in FIG. 15) that the properties of the ductile tantalum entirely dominate the physical properties of the particle.

The reticulated substrate diagrammatically illustrated in FIG. 12 and the enlarged FIG. 13 may be produced by projecting laser beams or collimated light (for example ultraviolet light) through a pool of monomer that cross links when irradiated with such light. The shapes and sizes of the resulting pores are closely controlled and generally substantially uniform, so the properties of the substrate are generally approximately constant across the entire billet. When carbonized and coated with a ductile metal, the resulting reticulated composite may have properties, particularly structural properties, that are different from those exhibited by spheroidal pored composites.

FIG. 14 serves to graphically illustrate the structure that is described as pore or cell 10, and window 12. The image is of a reticulated composite with a tantalum coating on a vitreous carbon skeleton where the tantalum is present in the amount of approximately 14 volume percent.

FIG. 15 shows the stress-strain plot for a conventional ductile tantalum coated reticulated vitreous carbon substrate wherein the tantalum is present in the amount of about 14 volume percent. The physical properties of this composite are dominated by those of the ductile tantalum. This is shown by the upward trend of the curve after the plateau that appears between approximately 7 and 20 percent strain. This material is not workable to form caltrop shaped particles, and it is not manually workable. The ductile tantalum undergoes ductile deformation rather than fracturing.

By contrast to FIG. 15, as shown by the curves in FIGS. 16 and 17, composites with ductile tantalum amounts of from approximately 1 to 5 volume percent reach a plateau and then crush with the application of further loads. The plots in FIGS. 16 and 17 are in terms of stress versus inches of movement of a crushing element in the test apparatus, rather than stress versus strain as shown in FIG. 15. The shapes of the curves in FIGS. 16 and 17 are the same as those plotted in conventional stress-strain plots such as FIG. 15. In the tests reflected in FIG. 16, the sample with a tantalum coating of about 0.67 volume percent had a plateau strength of approximately 65 pounds per square inch (psi), and the sample with about 1.06 volume percent tantalum had such a plateau strength of approximately 110 psi. In the tests reflected in FIG. 17, the sample with a tantalum coating of about 3.71 volume percent had a plateau strength of approximately 550 psi, the sample with about 3.89 volume percent tantalum had such a plateau strength of approximately 700 psi, and the sample with about 4.50 volume percent had a plateau strength of about 800 psi. The samples reflected in FIG. 17 were easy to manually work with a Dremel tool. It was possible but difficult to obtain a satisfactory yield of caltrop shaped particles from these samples.

FIG. 18 is an image of a sample with approximately 3 volume percent tantalum that was subjected to a crush test. It shows some mushrooming indicating some ductility even though the shape of the stress-strain plot for this sample closely resembled those of the samples reflected in FIGS. 16 and 17. It would be possible to manually shape this billet, and to reduce it to an acceptable yield of caltrop shaped particles.

The biocompatibility and mechanical properties of particular embodiments are generally determined by an iterative process. In practicing this iterative process, for example, approximately 1 volume percent of ductile tantalum or other ductile metal is applied to the surfaces of a brittle reticulated substrate. Microscopic examination is generally used to confirm that the coating is continuous. The resulting reticulated tantalum composite billet is then crushed at a measured speed to measure its fracture strength in compression. A Dremel tool, saw, or other tool is used to determine if the monolithic reticulated composite billet can be manually shaped by a surgeon in an ordinary operating room environment without disintegrating. Size reduction techniques such as milling, machining, crushing, screening, or the like, are then used to determine if at least a substantial amount of this billet can be converted into caltrop shaped morselized material of the desired particle size. If the resulting reticulated tantalum composite is too fragile to be handled in the operating room, additional tantalum is deposited until the desired strength is achieved. If it undergoes mostly plastic deformation instead of fracturing, the amount of tantalum is decreased. If it cannot be manually worked, the amount of tantalum is reduced. The desired tantalum loading may also be approached from the high end by starting with, for example 10 to 15 volume percent tantalum. If the resulting reticulated composite billet exhibits too much deformation strength, is difficult to shape manually, or is difficult to convert into morselized material, another reticulated tantalum composite is formed with slightly less tantalum. The process is repeated until the desired results are achieved. The composition of a composite billet with the optimum properties for a particular purpose is arrived at by an iterative process. With a particular end use in mind, successive billets may be produced by those skilled in the art according to the teachings herein, and tested. For example, different volume percentages of brittle substrate and ductile metal coating may be tried, or different metals or substrates may be tried. The best method of production and composition of a composite billet for a particular intended purpose may thus be determined by one skilled in the art following the teachings herein. The proper strength of the metal is generally determined by an iterative process following the teachings herein, with careful attention being paid to maintain the biocompatibility of the composite.

Once a reticulated tantalum or other ductile metal composite with the desired degree of brittleness and strength is obtained, the biocompatibility of the composite may be determined by in vitro, and, if necessary, in vivo procedures. If the desired degree of biocompatibility is not achieved, then the proportion of the tantalum or other ductile metal in the composite may be adjusted until it is achieved. If necessary, the optimum desired physical properties of strength and brittleness may be compromised somewhat to achieve the best biocompatibility. There will generally be a range of fracture toughness, ductility, and strength within which the reticulated tantalum composite is satisfactory for its intended purpose.

When intended for use as a precursor to morselized particulates, embodiments of these reticulated composites should have sufficient fracture strength so that the morselized particulates do not fracture into smaller particles of unknown size and shape during normal handling and use in surgical procedures. Embodiments that are intended to be surgeon shapeable do not necessarily have the same fracture strengths as those embodiments that are intended to be reduced to morselized particles. Since morselization takes place in a manufacturing environment, and not an operating room environment, size reduction equipment may, for example, be adapted to accommodate fracture strengths that are higher than those that would be considered optimum for surgeon shapeable reticulated composites.

When certain embodiments of reticulated tantalum composites are reduced in size to morselized particles, the particles generally exhibit a form wherein several legs project generally outwardly from an intersection of those legs at a node in what may be described as a 3-dimensional, spiny, multi-axial form as shown, for example, in FIG. 5 and FIG. 19. These are typical caltrop shaped particles. Such particles tend to interlock with one another so that a mass of such caltrops tends to hold its shape against shear, compression, or tensile loads without the aid of adhesive or any other binding agent. This is advantageous when a mass of such caltrops are to be used to fill a cavity in a bone. Such caltrops may include enough intact ligaments to form one or more windows or pores. As the pore size of the substrate decreases, or the size of the morselized caltrops increases, the number of pores and windows in the caltrops tends to increase. In general, the average diameter of morselized caltrops ranges from approximately 0.1 to 10, or in further embodiments, approximately 0.2 to 2, or approximately 0.2 to 1 millimeters. For purposes of illustrating morsel sizes and scale, an embrittled (hydrided) 80 pore per inch reticulated tantalum-vitreous carbon composite was crushed and the resulting particles were classified by screening as indicated in FIG. 11.

Several samples of reticulated tantalum-vitreous carbon composite materials were prepared and tested. Reticulated vitreous carbon substrates with approximately 80 pore per inch (ppi) porosity were used. A monolithic billet of reticulated tantalum composite was prepared by infiltrating tantalum by CVI into the vitreous carbon substrate to achieve a tantalum content of about 2 volume percent. The billet had a carbon content of about 3 volume percent, and a void volume of about 95 percent. This billet was easily shaped by a handheld Dremel tool without disintegrating or undesired fracturing. Another billet was formed by infiltrating tantalum by CVI until about 4 volume percent of tantalum was achieved. This billet was easily shaped by a handheld Dremel tool, but shaping was not as easy as for the 2 volume percent. Another billet was formed by infiltrating tantalum by CVI until about 3 volume percent of tantalum was achieved, and it too was machinable with a Dremel. The billet containing approximately 2 volume percent tantalum was subjected to a size reduction operation to produce caltrop shaped morsels. The billet was machined with about a 1 millimeter deep cut on a conventional end mill using a 1 inch diameter, 2 flute bit rotating at 125 revolutions per minute with a lateral feed rate of about 12 inches per minute. Some of the resulting caltrop shaped morsels are shown in FIG. 19. These caltrop shaped morsels resulting from this size reduction ranged in size from approximately 250 to 1750 microns, with the majority of the particles in the approximate 500 to 1000 micron range. More than approximately 80 weight percent of the morsels exhibited a typical caltrop shape. A billet with a CVI deposited tantalum content of about 2 volume percent was also manually crushed through a 10 mesh sieve to yield a morselized composite material with the largest mass fraction of the particles being in the size range of −10 mesh to +40 mesh (0.4 to 2 millimeters).

Depending on the amount of tantalum present in a tantalum lean reticulated composite with a brittle substrate, the reticulated composite's compressive plateau strength is generally between approximately 50 and 1000 pounds per square inch (psi), and in some further embodiments, approximately 40 to 2000 psi, approximately 200 to 1200 psi, or approximately 100 to 1000 pounds per square inch. The compressive strength of the reticulated composite increases as the volume fraction of tantalum increases. At volume fractions of approximately 0.5 to 5 volume percent tantalum, approximately 3 volume percent vitreous carbon substrate, and the balance void space, the compressive plateau strengths of the respective reticulated composites range from approximately 50 to 1200 pounds per square inch. Reticulated composites with volume fractions of tantalum that exceed approximately 10 volume percent generally exhibit compressive plateau strengths in excess of 5000 pounds per square inch. The compressive strengths of conventional reticulated tantalum composites generally range from approximately 7,000 to 10,000 pounds per square inch for tantalum volume fractions in excess of approximately 14 percent.

The fracture strengths in compression of uncoated reticulated brittle substrates, such as vitreous carbon, that are suitable for use in practicing the present invention generally range from approximately 30 to 150 psi, and in further embodiments approximately 40 to 100 psi, or 40 to 60 pounds per square inch. The volume fraction of the vitreous carbon substrates is generally minimized, and the void volume is generally maximized, although reticulated vitreous carbon substrates in the amount of as much as approximately 1 to 10 volume percent, or in further embodiments, approximately 3 to 8 volume percent, or approximately 3 to 5 volume percent may be used.

Reticulated brittle monolithic substrates that are suitable for use according to the present invention include, for example, those made according to the teachings of Stankiewicz U.S. Pat. No. 6,103,149. Attention is invited, for example, to FIG. 6 of this patent, which illustrates one embodiment of those reticulated skeleton substrates that are suitable for use in practicing the present invention. Jacobsen, U.S. Pat. No. 7,382,959, which is hereby incorporated herein by reference as though fully set forth hereat, refers to a technique for making a reticulated monolithic structure comprised of polymer. Such polymeric monoliths may be converted to carbon using, for example, the teachings of Stankiewicz to form reticulated vitreous carbon skeletons that are suitable for use as substrates in practicing the present invention. The pores and windows in such skeletons are generally polyhedral and polygonal rather than spheroidal and circular, respectively.

Certain embodiments of reticulated skeleton substrates comprise a substantially monolithic 3-dimensional network of ligaments. Such ligaments generally extend in different planes and are interconnected at nodes to define a plurality of skeleton pores. Each ligament within such a network of ligaments generally extends between two nodes. Such networks of ligaments include a plurality of nodes. At the surfaces of the monolith, the ligaments are broken at or between nodes with the fracture surfaces being approximately coplanar with and defining the surface of the monolith. Such networks of ligaments form reticulated skeleton substrates that are generally monolithic and have typical void volumes of 97 volume percent, but can range from approximately 90 to 99 volume percent, although void volumes as low as about 66 volume percent may be used, if desired. A reticulated vitreous carbon skeleton generally comprises a three-dimensional network of ligaments interconnected at nodes to define generally spheroidal pores and generally circular windows between those pores. The generally spheroidal pores generally range in diameter from approximately 400 to 7500, and in further embodiments, from approximately 400 to 900 microns. The windows between these pores generally range in diameter from approximately 40 to 4500, and in further embodiments, approximately 40 to 500 microns in diameter. Such reticulated skeleton substrates are generally suitable for use in practicing the present invention. According to further embodiments, suitable reticulated vitreous carbon skeletons have from approximately 20 to 300, and in further embodiments, approximately 40 to 200, or approximately 60 to 100 pores per inch (ppi).

The materials of which useful reticulated skeleton substrates are composed generally have a stand-alone fracture strength in compression of less than approximately 150 pounds per square inch. This fracture strength being “stand-alone” in the sense that the strength of the skeleton is measured without any other material being present in or on the skeleton. Embodiments of these reticulated skeleton substrates have sufficient strength to permit them to be handled in further processing to form composites. In embodiments where the strength, fracture toughness, and ductility properties of the reticulated skeleton substrates are intended to dominate those of the other materials in the composite, the reticulated skeleton substrates must have sufficient fracture strength in compression to avoid unintended breakage during processing and handling of the composite. For example, in such composite embodiments that are intended for use as monolithic surgeon shapeable implants, the strength of the reticulated skeleton substrates should be at least approximately 10 pounds per square inch, and that of the composites based on such reticulated skeleton substrates should be at least 50 pounds per square inch. Where such composite embodiments are intended to be reduced in size and used as morselized implants, the strength of the reticulated skeleton substrates should also be at least approximately 10 pounds per square inch, and that of the composites based on such reticulated skeleton substrates should be at least 50 pounds per square inch. Because these materials tend to crush in a progressive manner, the stress-strain curves for these materials generally display a plateau stress, which corresponds to a failure of ligaments near the surface. Some plastic deformation of a composite billet with a reticulated carbon substrate may also take place.

The desired properties in a reticulated metal composite may not, in certain embodiments, be easily achievable utilizing just a brittle substrate and a single ductile metal deposit. Other materials that are not incompatible with the intended implant use may be present in the brittle substrate or deposited onto the brittle substrate in co-deposition or sequential deposition procedures. For example, an alloy of tantalum and niobium co-deposited on a graphite substrate material, sequential deposits of tantalum and titanium on a vitreous carbon substrate material, or sequential deposits of embrittled tantalum and ductile tantalum on a reticulated such substrates may be used.

Embodiments of reticulated skeleton substrates that will be suitable for use according to the present invention are composed of bio-compatible brittle materials, including, for example, vitreous carbon, graphite, pyrolitic carbon, and other forms of carbon; calcium carbonate, calcium phosphate, hydroxyapatite, and similar biocompatible materials. Metals that will be suitable for use as coatings on reticulated skeleton substrates according to the present invention include, for example, tantalum, titanium, niobium, mixtures and alloys thereof comprised substantially of such metals. Further embodiments include mixtures and alloys of such metals that exhibit reduced ductility when they incorporate light elements such as, for example, hydrogen or oxygen. The brittleness of these materials is indicated, for example, by the fact that, for any given material, their ability to undergo plastic deformation is minimal. The peak tensile stress noted on a stress-strain plot, and the ultimate tensile strength of that material are approximately the same, and/or the materials have very low fracture toughness. With low fracture toughness, once a fracture develops, these materials exhibit very little resistance to further propagation of the fracture. Since they are intended for use in implants, all of the materials in these reticulated composites are biocompatible.

The amount of the metal deposited in these composites, relative to the amount of the reticulated skeleton substrate, is such that the fracture behavior of these composites is sufficiently affected by the fracture properties of the brittle reticulated skeleton substrate as to make composite billets manually workable with tools that are amenable to use in an operating room. Such tools include handheld power tools as well as hand-powered tools such a knives and handsaws. When the proportionate amount of the metal coating is below the threshold needed to impart high strength, high fracture toughness, and ductility in the composite, the resulting reticulated metal composites have reduced strength, reduced fracture toughness, and reduced ductility.

For practical purposes, the significant characteristic that reflects all of strength, fracture toughness, and ductility for a composite reticulated billet is often manual workability. If a surgeon with ordinary hand strength can manually shape a billet with the tools that are customarily available in the conventional operating room without fracturing the billet in undesired areas, the strength, fracture toughness, and ductility have been achieved for the purposes of the present invention. If a billet is too fragile or weak to withstand manual shaping, it is not suitable for use according to the present invention. Where the intended purpose is to produce a morselized composite, a composite billet that is too strong to shape manually, may exhibit such low fracture toughness and ductility as to be capable of being machined to produce morsels without significant deformation.

Caltrop shaped particles are particularly desirable for use as morselized particles. The general caltrop shape tends to allow the morsels to interlock with one another when placed in a cavity in a bone. The structural support that this interlocking provides is particularly important during the healing process. For purposes of forming caltrop shaped particles from a composite billet, a size reduction operation is generally used. The fact that a composite can be reduced to such caltrop shaped particles is an indication that the billet exhibits acceptable strength, fracture toughness, and ductility characteristics for purposes of this invention. If at least most of a composite reticulated billet can be reduced to caltrop shaped composite particles with a size reduction operation, the necessary strength, fracture toughness, and ductility have been achieved for the purposes of the present invention. If a composite deforms rather than fracturing, or shatters into mostly powder, it is usually not usable according to the present invention. The term “caltrop” as used in this disclosure and the claims appended hereto is used in a general sense to include all spiny particles, including generally straight ligament sections that result from fracturing reticulated tantalum composites into morselized particles that tend to interlock with one another.

In general, if the strength, fracture toughness, and ductility properties are such that a billet disintegrates to dust when subjected to a size reduction operation, the volume fraction of metal should be increased. If a billet smears or undergoes undesirable levels of plastic deformation when subjected to size reduction operations or manual working, in general, the volume fraction of the metal should be reduced. Also, according to some embodiments, the ductility of the metal may be adjusted by adding or removing embrittling elements from it, provided the bio-compatibility of the composite is not adversely changed. In general, the metal may be strengthened somewhat if the brittleness of the metal is slightly increased.

In general, the tantalum or other biocompatible ductile metal has a stand-alone deformation tensile strength of at least approximately 20,000 pounds per square inch. That is, the ductile metal by itself, apart from the composites, exhibits such deformation tensile strength. The proportion of the metal deposit in these composites is sufficient to provide a biocompatible micro- and nano-textured surface that promotes ingrowth and adhesion of new bone, yet certain embodiments of these reticulated metal composites exhibit a fracture strength in compression of less than approximately 1000 pounds per square inch. These reticulated composites exhibit minimal plasticity because most ligaments break before they undergo substantial plastic deformation.

Some surgical applications in the bone graft area had previously required the use of morselized bone to fill a void in a patient's bone. Various synthetic morselized materials had been proposed for use in place of morselized bone. Ductile metals such as tantalum had not been considered for use as a replacement for morselized bone, because it is very difficult to form the necessary small particles of ductile metal that could serve as replacements for morselized bone. Reducing a body of a ductile metal such as tantalum into particles is difficult because of the strength and ductility of the metal. Reducing a reticulated monolithic body of ductile metal into particles is equally difficult because it deforms rather than fracturing. Thus, the substantial therapeutic advantages offered by the surface properties of ductile tantalum had not been available in morselized applications.

What has been described herein are preferred embodiments in which modifications and changes may be made without departing from the spirit and scope of the accompanying claims. While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A method of manufacturing comprising:
selecting a skeleton, said skeleton being reticulated, biocompatible, and substantially monolithic, said skeleton comprising a reticulated three-dimensional network of ligaments interconnected at nodes to define pores and windows between said pores, said skeleton having from approximately 3 to 300 pores per inch, a fracture strength in compression of less than approximately 150 pounds per square inch, and a void volume of at least approximately 66 percent;
forming a deposit of ductile biocompatible metal on said skeleton in an operation that comprises practicing a chemical vapor infiltration procedure to form a reticulated biocompatible composite, and carrying out said operation under conditions wherein said deposit exhibits a biocompatible surface adapted to promote bone ingrowth and adhesion, said deposit having a stand-alone deformation tensile strength of at least approximately 20,000 pounds per square inch; and
limiting said deposit to an amount at which said reticulated biocompatible composite has a fracture strength in compression of less than approximately 1000, or 1200, or 2000 pounds per square inch.

2. A method of manufacturing according to claim 1 wherein said deposit comprises tantalum.

3. A method of manufacturing according to claim 1 wherein said deposit comprises niobium.

4. A method of manufacturing according to claim 1 wherein said deposit comprises an alloy of tantalum or niobium.

5. A method of manufacturing according to claim 1 wherein said reticulated biocompatible composite has a fracture strength in compression of less than approximately 1000 pounds per square inch.

6. A method of manufacturing according to claim 1 wherein said reticulated biocompatible composite has a fracture strength in compression of less than approximately 2000 pounds per square inch.

7. Method of using a reticulated composite monolith comprising: commencing an in vivo surgical procedure on a living being that will require that a portion of the living being's bone be replaced by a customized scaffold;
selecting a said reticulated composite monolith that comprises a brittle biocompatible substrate with a coating of ductile metal that is biocompatible, and that includes a 3-dimensional network of ligaments interconnected at nodes forming pores and windows between said pores, said reticulated composite monolith having substantially no deformation strength, a fracture strength in compression of less than approximately 1000, or 1200, or 2000 pounds per square inch, and the biocompatible surface characteristics of said ductile metal; and
forming said customized scaffold by manually shaping said reticulated composite monolith during said in vivo surgical procedure.

8. Method of using a reticulated composite monolith according to claim 7 wherein said reticulated biocompatible composite has a fracture strength in compression of less than approximately 1200 pounds per square inch.

9. A method of using of claim 7 wherein said manually shaping includes fracturing said reticulated composite monolith to change its shape and expose said brittle biocompatible substrate where said reticulated composite monolith is fractured.

10. A method of using of claim 7 including selecting a said reticulated composite monolith that comprises a brittle biocompatible substrate comprised of vitreous carbon with a coating of ductile metal comprised of tantalum.

11. A method of using of claim 7 wherein said reticulated biocompatible composite has a fracture strength in compression of less than approximately 1000 pounds per square inch.

12. An article of manufacture comprising:
a reticulated composite monolith comprised of a 3-dimensional network of ligaments interconnected at nodes to define pores and windows between said pores, said reticulated composite monolith being comprised of biocompatible materials including a brittle substrate and a ductile metallic coating on said brittle substrate, said reticulated composite monolith being bicompatible and having a fracture strength in compression of less than approximately 1000, or 1200, or 2000 pounds per square inch, and said ductile metal coating having surface characteristics that promote bone growth and adhesion.

13. An article of manufacture of claim 12 wherein said reticulated composite monolith has a fracture strength in compression of less than approximately 1000 pounds per square inch.

14. An article of manufacture of claim 12 wherein said reticulated composite monolith has a fracture strength in compression of less than approximately 1000 pounds per square inch, said brittle substrate being comprised of vitreous carbon, and said ductile metal being comprised of tantalum.

15. An article of manufacture of claim 12 wherein said reticulated composite monolith has a fracture strength in compression of less than approximately 1200 pounds per square inch.

16. An article of manufacture of claim 12 wherein said reticulated composite monolith has a fracture strength in compression of less than approximately 2000 pounds per square inch.

17. An article of manufacture of claim 12 wherein said reticulated composite monolith comprises a skeleton of vitreous carbon coated with from approximately 1 to 10 volume percent of ductile tantalum, or from approximately 2 to 5 volume percent of ductile tantalum.

18. A reticulated composite monolith that is biocompatible comprising:
a skeleton, said skeleton comprising vitreous carbon and being substantially monolithic, said skeleton comprising a reticulated three-dimensional network of ligaments interconnected at nodes, said skeleton having a stand-alone fracture strength in compression of less than approximately 150 pounds per square inch;
a metal coating on said skeleton comprised of ductile biocompatible metal, said metal coating having a stand-alone deformation tensile strength of at least approximately 20,000 pounds per square inch and a submicron-textured surface that promotes ingrowth and adhesion of new bone, said metal coating being present in an amount sufficient to provide said submicron-textured surface, and proportioned relative to said skeleton such that said reticulated composite monolith has a fracture strength in compression of less than approximately 1000, or 1200, or 2000 pounds per square inch.

19. A composite caltrop that is biocompatible comprising:
a brittle substrate comprised of vitreous carbon having a stand-alone fracture strength in compression of less than approximately 150 pounds per square inch;
a metal coating on said brittle substrate comprised of ductile biocompatible metal, said metal coating having a stand-alone deformation tensile strength of at least approximately 20,000 pounds per square inch and a submicron-textured surface that promotes ingrowth and adhesion of new bone, said metal coating being present in an amount sufficient to provide said submicron-textured surface, and proportioned relative to said brittle substrate such that said composite caltrop has a fracture strength in compression of less than approximately 1000, or 1200 or 2000 pounds per square inch.

20. A composite caltrop according to claim 19 wherein said composite caltrop has peripheral surfaces, at least some of which are broken, and said brittle biocompatible substrate is exposed where said peripheral surfaces are broken.

21. A composite caltrop according to claim 19 wherein said brittle bicompatible substrate is comprised of vitreous carbon, and said metallic coating is comprised of tantalum.
